(19) United States
(12) Patent Application Publication
(10) Pub. No.: US 2005/0164325 A1
(43) Pub. Date: Jul. 28, 2005

(54) Method of mass spectrometry

(75) Inventor: Stephen Leicester, Manchester (GB)

Correspondence Address: WATERS INVESTMENTS LIMITED;C/O WATERS CORPORATION, 34 MAPLE STREET - LG, MILFORD, MA, 01757, US

(73) Assignee: Micromass UK Limited, Manchester (GB)

(21) Appl. No.: 10/947,833

(22) Filed: Sep. 23, 2004

Related U.S. Application Data
(60) Provisional application No. 60/506,757, filed on Sep. 30, 2003.

(30) Foreign Application Priority Data
Sep. 24, 2003 (GB) 0322356.7

(51) Int. Cl. C12Q 1/37; C12P 21/06; G01N 33/00

(52) U.S. Cl. 435/23; 435/68.1; 436/86

(57) ABSTRACT

A method of identifying post-translationally modified proteins is disclosed. The method comprises mass analysing peptide ions and then subtracting from the determined mass of the peptide ion the known increase in mass due to one or more modifications of interest. The resulting value which represents the mass a peptide would have, had the protein from which it is derived not been modified, is then used to search against a peptide databank. A short list of possible peptides is formed by selecting peptides which have both the right mass or mass to charge ratio (to within a user specified tolerance) and which also support at least one of the user selected modifications of interest. Each short listed peptide is then scored in turn against fragmentation data related to the experimentally observed peptide.

### CROSS REFERENCE TO RELATED APPLICATIONS

This application claims priority from United Kingdom patent application GB-0322356.7, filed 24 Sep. 2003 and U.S. Provisional Application 60/506,757, filed 30 Sep. 2003. The contents of these applications are incorporated herein by reference.

### FIELD OF THE INVENTION

The present invention relates to a method of identifying a modified protein, a mass spectrometer comprising means for identifying a modified protein and an apparatus for identifying a modified protein.

### BACKGROUND OF THE INVENTION

The genetic information stored in DNA defines the sequence of amino acids in polypeptides (proteins). The genetic code can be considered to comprise the set of rules by which DNA nucleotide base pair sequences are translated into a corresponding sequence of amino acids. A code word (codon) for an amino acid consists of a sequence of three nucleotide base pairs. The sequence of base pairs in DNA is first transferred to information-transmitting messenger RNA (mRNA) molecules in a process called transcription. These then serve as the template for synthesis of polypeptides in a process called translation. Translation occurs in ribosomes in the cytoplasm. The polypeptide chains so formed leave the ribosome in an “unmodified” form. However, the polypeptide chains can then become modified in a variety of ways. Some such as, for example, those containing the amino acid cysteine may modify themselves by folding into a certain shape so that they can form di-sulphide bonds between cysteins. This governs the shape of the protein and often it is the shape of the protein that determines its function. Others may become modified by the chemical derivitisation of certain functional groups on specific amino acids. For example, the functional group on the amino acid tyrosine can become phosphorylated. This changes the polarity and shape of the polypeptide and can enable it or disable it from performing certain functions. The above are examples of post-translational modifications which occur after the polypeptide has been synthesised and when the polypeptide is freely mixing with other polypeptides or chemicals. There are numerous forms of post-translational modifications nearly all involving some form of chemical derivitisation of a functional group. They include methylation, hydroxylation, oxidation, formylation, acetylation, carboxylation, phosphorlyation, sulphation, cysteinylation, glycosalation, farnesylation, myristoylation, biotinylation, palmitoylation and stearoylation. Some of the modifications are more common than others.

The ability to be able to identify proteins (commonly referred to as “protein characterisation”) is of particular interest in the life sciences field. Protein identification may involve recognising a protein by comparing experimental data with data held in a protein database. Alternatively, protein identification may involve working out ab-initio the sequence of amino acids which form the protein.

The ability to be able to identify or characterise proteins which have been post-translationally modified is of particular interest. Mass spectrometry is commonly used as a means of identifying proteins and post-translationally modified proteins.

It is known to compare experimental mass spectral data which has been obtained by mass analysing peptide samples with theoretical peptide data generated from protein sequence or Expressed Sequence Tag (EST) databanks. Theoretical peptide data can be obtained by predicting the peptides which would be formed when proteins listed in the protein databank are digested. Digest rules can be applied to one or more of the proteins listed in the protein databank and then the mass to charge ratio of the resulting peptide products can be determined.

In addition to comparing experimental mass spectral data against theoretical peptide data, it is also known to use fragmentation (or MS/MS) data to identify peptides and the protein from which they are derived. Peptide ions when subjected to high energy collisions in a collision or fragmentation cell of a mass spectrometer will fragment into a number of fragment ions which can then be mass analysed. The process by which peptide ions fragment can also be simulated starting from the peptides which are theoretically expected from the digestation of a protein listed in the protein databank. Theoretical fragmentation data obtained by predicting how peptides may fragment can then be compared against fragmentation mass spectral data obtained experimentally.

The use of fragmentation or MS/MS data is particularly powerful in protein characterisation (and especially proteins which have been post-translationally modified) since the fragment ions formed due to the fragmentation of a peptide ion will depend both upon the location and type of any post-translational modifications which have affected the protein.

Early techniques of comparing experimental mass spectral data against theoretical peptide data involved directly comparing experimental mass spectral data with peptide data held in an indexed pre-digested peptide databank. This approach was suitable for identifying unmodified proteins but did not allow post-translationally modified proteins to be identified since the peptide databank did not support proteins which had been post-translationally modified.

The increasing need to support protein characterisation and especially post-translationally modified proteins has meant that a different approach has had to be adopted in order to identify post-translationally modified proteins. It has not previously been considered practical to search peptide data derived from a digested post-translationally modified protein against conventional indexed pre-digested peptide databanks since details of each unmodified peptide stored in the peptide databank would also need to be complemented by numerous additional entries relating to all potential combinations of modifications and the various permutations of the location of such modifications. Any peptide databank which included all possible modifications and permutations of modifiable sites would be so large that it would be impractical to use as search times for searching such a databank would be very slow.

In view of the impracticality of attempting to use a peptide databank which includes entries for all possible modifications to a protein, the conventional approach to identifying post-translationally modified proteins is to start instead with a conventional protein databank (as opposed a pre-digested peptide databank). Proteins listed in the protein databank are then theoretically digested on the fly i.e. the proteins are theoretically digested each time experimental peptide data is submitted to be analysed.

According to the conventional approach, any particular modifications which a user wishes to consider are then applied completely or in part to the peptide products which result from the theoretical digestion of the protein. The resultant theoretically modified peptide data can then be compared with the actual experimental mass spectral data. The conventional approach therefore allows searches to support any number of potential (i.e. variable or random) modifications. However, one of the drawbacks of the current approach is that it takes a relatively long time to theoretically digest proteins listed in a protein databank. Also, since the theoretical digestion of proteins listed in a protein databank takes place on the fly, every peptide thus generated needs to be analysed for all combinations of modifications which the user wishes to consider. Furthermore, there are potentially numerous different permutations of modifiers which may need to be considered. For example, if a peptide has several modifiable sites and a single modification is considered, then each modifiable site must be considered in turn when generating the theoretical peptide data and when looking for the best (combination and permutation) fit to the experimental data. Consequently, the conventional approach is relatively slow especially when a number of modifiers are to be considered.

It is therefore desired to provide a faster method of identifying or characterising a protein which has been post-translationally modified.

### SUMMARY OF THE INVENTION

According to the present invention there is provided a method of identifying a modified protein comprising:
digesting a modified protein to produce a plurality of peptides;
ionising at least one of the plurality of peptides to form one or more peptide ions;
mass analysing one or more of the peptide ions to determine the mass or mass to charge ratio of at least one of the peptides or peptide ions; and
identifying the modified protein by:
(i) determining the theoretical unmodified mass or mass to charge ratio which a peptide or peptide ion would have, had the protein from which the peptide or peptide ion is derived not been modified;
(ii) searching a databank of peptides using the theoretical unmodified mass or mass to charge ratio; and
(iii) determining peptides in the databank which have a mass or mass to charge ratio which corresponds with the theoretical unmodified mass or mass to charge ratio.

The modified protein has preferably been post-translationally modified e.g. by methylation, hydroxylation, oxidation, formylation, acetylation, carboxylation, phosphorlyation, sulphation, cysteinylation, glycosalation, farnesylation, myristoylation, biotinylation, palmitoylation or stearoylation.

One or more modifications of interest are preferably determined, selected or otherwise considered. The modified protein is preferably determined or considered to have been modified by at least one of the modifications of interest. At least one or more (preferably all) combinations of modifications of interest are preferably enumerated. A maximum number of modifications is preferably determined, selected or considered. Preferably, the step of enumerating through combinations of modifications of interest comprises enumerating through combinations of modifications of interest up to the maximum number of modifications.

The step of determining the theoretical unmodified mass or mass to charge ratio preferably comprises calculating or determining the mass or mass to charge ratio of a combination of modifications of interest. The theoretical unmodified mass or mass to charge ratio is preferably obtained by altering, reversing, counteracting, negating, unmodifying or adjusting the mass or mass to charge ratio of an experimentally observed peptide or peptide ion in view of the combination of modifications of interest. In particular, this may involve subtracting (or less preferably adding) the mass or mass to charge ratio of the combination of modifications of interest from (to) the mass or mass to charge ratio of the peptide or peptide ion as determined by the step of mass analysing.

The method preferably further comprises determining, selecting or considering an allowed mass or mass to charge ratio tolerance between the theoretical unmodified mass or mass to charge ratio of the peptide or peptide ion and the mass or mass to charge ratio of peptides listed in the databank. The mass tolerance may fall within a range selected from the group consisting of: (i) <0.01 Da; (ii) 0.01-0.02 Da; (iii) 0.02-0.03 Da; (iv) 0.03-0.04 Da; (v) 0.04-0.05 Da; (vi) 0.05-0.06 Da; (vii) 0.06-0.07 Da; (viii) 0.07-0.08 Da; (ix) 0.08-0.09 Da; (x) 0.09-0.10 Da; and (xi) >0.10 Da. The mass to charge ratio tolerance may fall within a range selected from the group consisting of: (i) ≦0.1 mass to charge ratio units; (ii) ≦0.01 mass to charge ratio units; (iii) ≦0.001 mass to charge ratio units; (iv) ≦0.0001 mass to charge ratio units; (v) ≦0.00001 mass to charge ratio units; and (vi) ≦0.000001 mass to charge ratio units.

The step of searching a databank of peptides preferably comprises determining or selecting peptides listed in the databank which have a mass or mass to charge ratio corresponding to the theoretical unmodified mass or mass to charge ratio to within the mass or mass to charge ratio tolerance.

The method preferably further comprises determining a peptide in the databank of peptides which has a mass or mass to charge ratio corresponding to the theoretical unmodified mass or mass to charge ratio to within an upper limit of the mass or mass to charge ratio tolerance. Similarly, the method preferably further comprises determining a peptide in the databank of peptides which has a mass or mass to charge ratio corresponding to the theoretical unmodified mass or mass to charge ratio to within a lower limit of the mass or mass to charge ratio tolerance. Peptides in the databank of peptides having masses or mass to charge ratios between the upper limit and the lower limit are preferably selected and preferably form an initial list of peptides.

Peptides in the initial list of peptides which do not actually support at least one of the combinations of modifications of interest are preferably rejected, discarded or assigned a low priority thereby forming a shortlist of possible peptides. As a result preferably at least a majority of peptides in the shortlist of possible peptides have: (i) a mass or mass to charge ratio which corresponds to the theoretical unmodified mass or mass to charge ratio of the peptide or peptide ion to within the mass or mass to charge ratio tolerance; and (ii) at least the same number and type of modifiable sites in order to support at least one of the combination of modifications of interest.

In the preferred method it is determined whether the modifiable sites of a peptide in the shortlist of possible peptides exactly matches a combination of modifications of interest. If the modifiable sites of a peptide in the shortlist of possible peptides exactly or substantially matches a combination of modifications of interest then the peptide in the shortlist is scored against fragmentation or MS/MS mass spectral data corresponding to the peptide ion. Scoring involves determining, evaluating or calculating the closeness of fit between theoretical data and experimental data. If, however, the modifiable sites of a peptide in the shortlist of possible peptides do not exactly or substantially match a combination of modifications of interest then the modifications of interest are grouped according to the residue to which they apply. Combinations of modifiable sites are then preferably enumerated and modifications of interest permuted amongst the modifiable sites, preferably on a per group basis. Each permuted peptide is then preferably scored or correlated against fragmentation or MS/MS mass spectral data corresponding to the peptide ion.

The databank preferably comprises a databank of peptides derived from unmodified proteins. The peptides listed in the databank are preferably arranged in order of mass or mass to charge ratio. In particular, the peptides may be arranged in order of monoisotopic mass or mass to charge ratio.

The step of identifying the modified protein preferably further comprises repeating steps (i), (ii) and (iii) for a plurality of different peptides or peptide ions.

According to another aspect of the present invention there is provided a mass spectrometer comprising:
an ion source for ionising at least one of a plurality of peptides derived from the digestion of a modified protein to form one or more peptide ions;
a mass analyser for mass analysing one or more of the peptide ions to determine the mass or mass to charge ratio of at least one of the peptides or peptide ions; and
identifying means for identifying the modified protein, wherein, in use, the identifying means:
(i) determines the theoretical unmodified mass or mass to charge ratio which a peptide or peptide ion would have, had the protein from which the peptide or peptide ion is derived not been modified;
(ii) searches a databank of peptides using the theoretical unmodified mass or mass to charge ratio; and
(iii) determines peptides in the databank which have a mass or mass to charge ratio which corresponds with the theoretical unmodified mass or mass to charge ratio.

According to another aspect of the present invention there is provided a method of identifying a modified protein, comprising:
(i) determining the theoretical unmodified mass or mass to charge ratio which a peptide or peptide ion would have, had the protein from which the peptide or peptide ion is derived not been modified;
(ii) searching a databank of peptides using the theoretical unmodified mass or mass to charge ratio; and
(iii) determining peptides in the databank which have a mass or mass to charge ratio which corresponds with the theoretical unmodified mass or mass to charge ratio.

According to another aspect of the present invention there is provided apparatus for identifying a modified protein, wherein, in use, the apparatus:
(i) determines the theoretical unmodified mass or mass to charge ratio which a peptide or peptide ion would have, had the protein from which the peptide or peptide ion is derived not been modified;
(ii) searches a databank of peptides using the theoretical unmodified mass or mass to charge ratio; and
(iii) determines peptides in the databank which have a mass or mass to charge ratio which corresponds with the theoretical unmodified mass or mass to charge ratio.

According to another aspect of the present invention there is provided a method of identifying a modified protein, comprising:
(a) determining, selecting or considering one or more modifications of interest;
(b) enumerating through one or more combinations of modifications of interest;
(c) calculating the mass or mass to charge ratio of one or more of the combinations of modifications of interest;
(d) subtracting the calculated mass or mass to charge ratio of one of the combinations of modifications of interest from the experimentally determined mass or mass to charge ratio of a peptide or peptide ion to determine a theoretical unmodified mass or mass to charge ratio which a peptide or peptide ion would have, had the protein from which the peptide or peptide ion is derived not been modified;
(e) searching a databank of peptides using the theoretical unmodified mass or mass to charge ratio to return an initial list of peptides;
(f) discarding from the initial list of peptides, peptides which cannot support the combination of modifications of interest thereby producing a short list of peptides; and
(g) scoring peptides in the short list of peptides against fragmentation mass spectral data.

According to another aspect of the present invention there is provided apparatus for identifying a modified protein, wherein, in use, the apparatus:
(a) determines, selects or considers one or more modifications of interest;
(b) enumerates through one or more combinations of modifications of interest;
(c) calculates the mass or mass to charge ratio of one or more of the combinations of modifications of interest;
(d) subtracts the calculated mass or mass to charge ratio of one of the combinations of modifications of interest from the experimentally determined mass or mass to charge ratio of a peptide or peptide ion to determine a theoretical unmodified mass or mass to charge ratio which a peptide or peptide ion would have, had the protein from which the peptide or peptide ion is derived not been modified;
(e) searches a databank of peptides using the theoretical unmodified mass or mass to charge ratio to return an initial list of peptides;
(f) discards from the initial list of peptides peptides which cannot support the combination of modifications of interest thereby producing a short list of peptides; and
(g) scores peptides in the short list of peptides against fragmentation mass spectral data.

The preferred method of identifying a modified protein involves directly searching against an indexed pre-digested unmodified peptide databank. Conventionally, it was not considered possible to search peptide data relating to a post-translationally modified protein directly against a peptide databank relating to unmodified proteins. However, the preferred embodiment shows that this assumption is incorrect.

Advantageously, the preferred embodiment allows any number of variable (random) modifications to be considered and hence is just as powerful as conventional approaches. However, since the preferred embodiment does not involve theoretically digesting proteins listed in a protein databank on the fly and then indexing them it is considerably quicker.

An important aspect of the preferred embodiment is that the mass or mass to charge ratio of an experimentally observed peptide is altered, reversed or otherwise “unmodified” to counteract or negate the effect that a particular post-translational modification has had on the mass of the peptide. The majority of post-translational modifications of interest have the effect of increasing the mass or mass to charge ratio of the protein and hence the mass or mass to charge ratio of various peptides derived from the protein will also be increased. An exception to this is dehydration which has the effect of reducing the mass or mass to charge ratio of a protein.

The step of counteracting, reversing or negating the effect of a post-translational modification involves determining the mass or mass to charge ratio which an experimentally measured peptide would have had, had the corresponding protein from which the peptide is derived not been subject to a post-translational modification. The preferred method therefore assumes variable or random modifications to have occurred to the protein (and hence affected the resulting peptides) and searches a conventional indexed pre-digested peptide databank of unmodified peptides using the calculated mass or mass to charge ratio of the unmodified peptide i.e. with the effects of possible modifications reversed.

In order to reduce overall search times as much as possible, the preferred embodiment filters possible peptides (i.e. peptides listed in the peptide databank which have a mass or mass to charge ratio which corresponds to the mass or mass to charge ratio of the experimentally observed peptide as subsequently unmodified) at high speed. This enables peptides listed in the peptide databank which may have the correct mass or mass to charge ratio but which can not actually support the modifications of interest to be ruled out or discarded. This ensures that those short listed peptides which are passed by the filtering process have suitable modifiable sites for the modifications of interest to have acted upon. In this way the preferred embodiment is able to determine rapidly those peptides which should then be considered as possible matches for the experimental peptide data.

Once peptides have been filtered to produce a short list of possible peptides, these possible peptides are then analysed in a second characterisation stage and scored against the experimental data.

Conventionally, for every experimentally observed peptide which is to be analysed, a theoretical digest of one or more proteins listed in a protein databank needs to be performed and indexed. The time taken to theoretically digest proteins listed in a protein databank can contribute a large proportion of the overall search time even though efficient algorithms are known for determining the digest products of a protein. Furthermore, every peptide generated by the theoretical digestion of a protein then needs to be processed by applying combinations of user selected random modifiers (if any can be applied) to a given peptide and then permuting the modification(s) amongst all the different possible modifiable sites on the peptide in question. This is also computationally time consuming and further contributes to relatively slow overall search times which are characteristic of the conventional methods of identifying post-translationally modified proteins.

The preferred method of identifying a modified protein has two important advantages over conventional approaches. Firstly, according to the preferred embodiment, it is not necessary to theoretically digest a protein listed in a protein databank or to index the peptide list, and hence these relatively time consuming steps can be avoided. Secondly, the number of possible peptides that need to be characterised can be reduced or otherwise restricted in a rapid manner.

Although not essential to the present invention, the preferred embodiment is particularly advantageous when accurate mass spectral data is used. This is achievable, for example, by using a hybrid quadrupole Time of Flight mass spectrometer and enables significantly faster overall search times to be obtained. For example, typical search times according to the preferred embodiment may be at least ×8 faster than conventional searches when using accurate mass spectral data. The use of accurate mass spectral data enables peptide filtering to be particularly effective in reducing the number of possible peptides which then need be considered or characterised.

### DESCRIPTION OF THE DRAWINGS

Various embodiments of the present invention will now be described, by way of example only, and with reference to the accompanying drawings in which:

FIG. 1 shows a number of different possible post-translational modifications which may affect a protein, the mass change resulting from such a modification and the peptide site to which such modifications apply;

FIG. 2 illustrates the steps taken according to the preferred embodiment in a first stage which involves unmodifying the mass or mass to charge ratio of experimentally observed peptides, searching a peptide databank for peptides which match the mass or mass to charge ratio of the unmodified peptide to within a specified tolerance and then filtering possible peptides to rule out peptides which are incapable of supporting the modifications of interest to produce a short list of possible peptides which have both the correct mass or mass to charge ratio and which also theoretically support the modification(s) of interest;

FIG. 3 illustrates the subsequent steps taken according to the preferred embodiment in a second stage wherein peptides which have been short listed in the previous filtering stage are scored against experimental fragmentation data;

FIG. 4 shows the number of combinations and binary searches performed when 168 peptides were analysed and different numbers of modifications and modifiable sites were considered;

FIG. 5 illustrates in more detail the number of peptides filtered and subsequently characterised and the associated time taken to filter, permute and score peptides against experimental data when considering two modifications;

FIG. 6 illustrates in more detail the number of peptides filtered and subsequently characterised and the associated time taken to filter, permute and score peptides against experimental data when considering four modifications; and

FIG. 7 shows comparative results for conventional search times against search times achieved according to the preferred embodiment and illustrates the improvement in overall search time which is achievable according to the preferred embodiment.

### DETAILED DESCRIPTION OF THE DRAWINGS

The rapid identification of proteins using mass spectral data and protein databanks is now a standard technique in proteomics work. As this area of research has developed, the ability to characterise proteins and in particular the ability to determine the presence and location of any post-translational modifications to the protein has become increasingly important. The preferred embodiment relates to a method of identifying post-translationally modified proteins more quickly than conventional techniques.

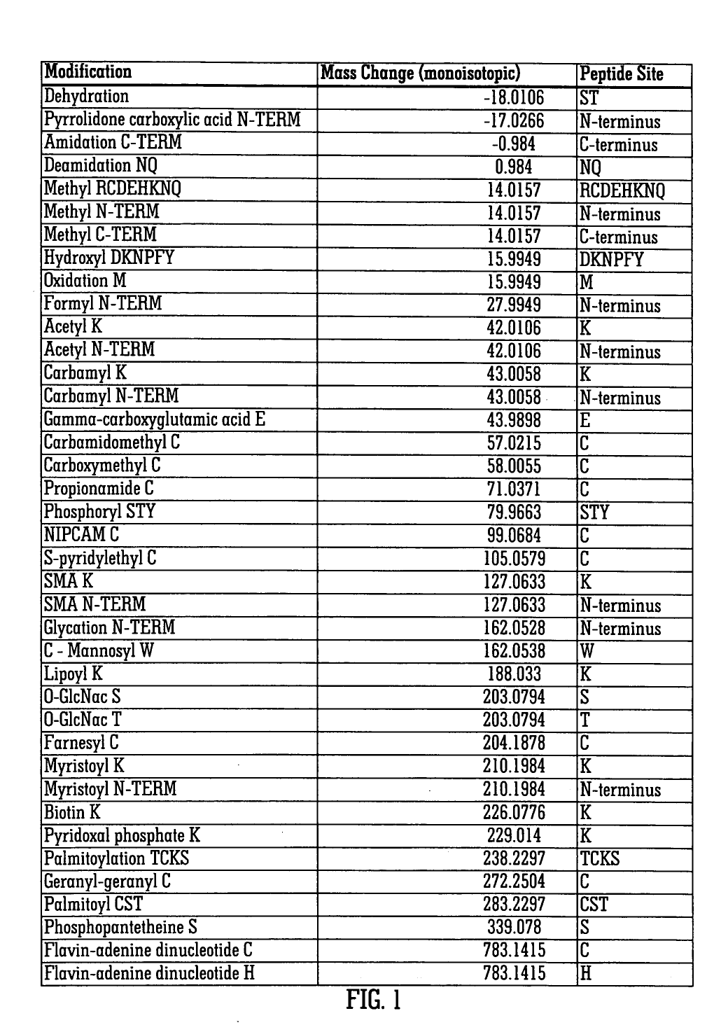

FIG. 1 shows a list of post-translational modifications which may affect a protein and the peptide sites or residues to which such modifications apply. The figure also shows the resulting change (normally increase) in mass to the protein caused by the modification.

It is known to subject peptides derived from digesting a protein to fragmentation in a collision of fragmentation cell of a mass spectrometer. The resulting fragmentation or MS/MS mass spectral data can then be analysed by searching against data held in a databank or against data derived from data held in a databank. One or more variable modifications may be selected and considered during such a search.

It has not, however, been considered practically possible to search for modified peptides using a conventional pre-digested peptide databank which only lists unmodified peptides. In order to enable such a search to be performed it has been assumed that the peptide databank would have to allow for all possible potential combinations of modifications and their permutations on a peptide. As a result it was thought that the peptide databank would need to be extensively increased with the result that the databank would then become too large and impractical to search against i.e. search times would be very slow.

For this reason, the conventional approach has been not to attempt to use pre-digested peptide databanks but rather to start from using a protein databank and then to theoretically digest proteins listed in the protein databank on the fly. Thereafter, variable modifications can be applied to the peptides determined to result from the digestion process. The resulting theoretically modified peptides can then be compared against the real experimental peptide data. In particular, selected peptides may be fragmented in a fragmentation cell of a mass spectrometer and the resulting fragmentation mass spectral data compared against theoretical fragmentation data obtained by theoretically fragmenting peptides resulting from the theoretical digestion of a protein listed in the protein databank. According to this conventional approach each protein in the protein databank is digested in turn and the process is then repeated.

In contrast to the conventional approach, the preferred embodiment makes use of an indexed pre-digested unmodified peptide databank when searching in respect of peptides which have been derived from a protein which is believed to have been post-translationally modified. Such an approach therefore avoids the need to digest proteins listed in a protein databank on the fly. Variable modification searches can still be carried out relatively quickly. According to the preferred method accurate mass spectral data acquired from, for example, a quadrupole Time of Flight mass spectrometer enables a high-speed peptide filter to be implemented. This enables search times to be significantly reduced and enables a more rapid means of protein characterisation.

The unmodified peptides listed in the peptide databank which is searched against are preferably sorted and arranged in monoisotopic mass order in order to facilitate databank searching. Importantly, the preferred embodiment involves searching directly against unmodified peptides stored in the peptide databank. Therefore, it is not necessary to attempt to provide multiple entries per peptide in the peptide databank in order to cover numerous different possible modifications which may be considered. The use of a databank of pre-digested unmodified peptides considerably simplifies the searching process.

Searching against the databank of unmodified peptides preferably involves two main sequential stages—a first filtering stage followed by a second characterising stage.

In the first stage an initial list of possible peptides is produced. All the peptides in this initial list have a mass or mass to charge ratio which matches that of the experimentally observed peptide (as unmodified according to the preferred embodiment) to within a user defined tolerance. This initial list is then preferably filtered to produce a short list of possible candidate peptides. This filtering process involves removing from the initial list all peptides which are incapable of supporting the modifications of interest. All the peptides in the resulting short list will therefore preferably have peptide sites which support the considered modifications.

Peptides in the short list of possible peptides are then processed in a second stage which relates to peptide characterisation (identification and location of modifications) and each shortlisted peptide is scored against fragmentation data relating to the experimentally observed peptide which is being analysed.

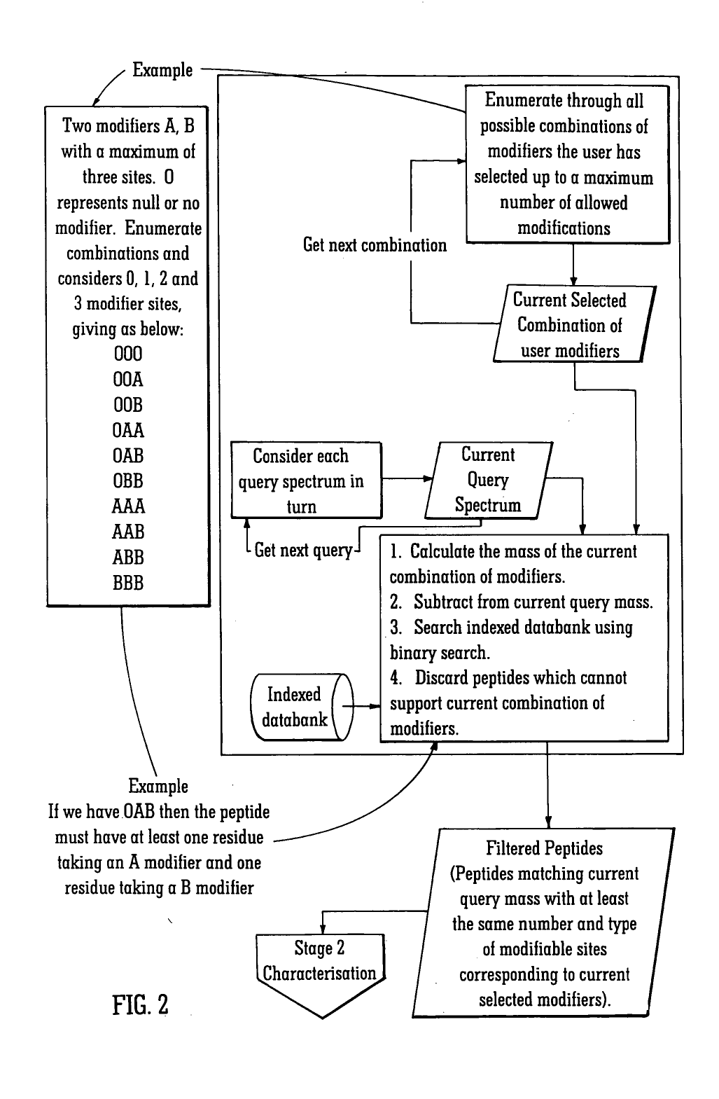

The first high speed peptide filtering stage as implemented according to the preferred embodiment is shown and described in more detail in relation to FIG. 2.

According to the preferred embodiment, different combinations of user selected modifiers are taken in turn and applied to the data corresponding to an experimentally observed peptide which is being analysed. According to the preferred embodiment it is assumed that an experimentally observed peptide is derived from a protein which has been post-translationally modified. Accordingly, the mass or mass to charge ratio of the experimentally observed peptide is adjusted or “unmodified” to take into account one or more assumed modifications. For example, if phosphorylation of serine is one of the modifiers or modifications which is of interest, then a single phosphoryl modifier (H₂PO₃) will be considered having enumerated through different combinations. If an experimentally observed peptide is assumed to carry a single phosphorolated serine then its mass will have been increased by 79.9663 as a result. Accordingly, in order to search against the databank of pre-digested unmodified peptides, the mass of the experimentally observed peptide is adjusted or unmodified to take account of the phosphorylation of the protein by subtracting the value of 79.9663 from the experimentally determined mass.

The above described approach of adjusting or unmodifying the experimentally determined mass or mass to charge ratio and searching against the peptide databank using the adjusted or unmodified peptide mass or mass to charge ratio is an important aspect of the preferred embodiment. This approach then enables a search to be made against a conventional indexed pre-digested peptide databank whilst enabling variable modifications to be considered. Having altered the mass or mass to charge ratio of the experimentally observed peptide to account for one or more assumed modifications, the indexed peptide databank can now be searched using the new (unmodified) theoretical mass of the peptide. The output from such a search of the peptide databank will comprise an initial list of possible peptides which have a mass or mass to charge ratio which matches that of the experimentally observed peptide (as unmodified according to the preferred embodiment) to within a certain tolerance. This initial list of peptides is then preferably filtered to provide a short list of candidate peptides. All the peptides in the short list will preferably support the desired combination of modifiers. In other words, the short list of possible peptides will comprise a list of peptides which all have at least the correct number of modifiable sites and wherein the modifiable sites will be of the correct type. Continuing with the above example relating to a single phosphoryl modifier, after the filtering stage, the peptides in the short list of possible peptides will all have at least one serine residue and will also have a mass or mass to charge ratio which substantially matches that of the observed peptide (as unmodified according to the preferred embodiment).

As the peptide data in the peptide databank is preferably sorted or arranged in mass order, all the possible peptides that have similar masses or mass to charge ratios to the mass or mass to charge ratio of an experimentally observed peptide (as unmodified according to the preferred embodiment) can be obtained from the peptide databank using two binary searches. The two binary searches correspond to the mass or mass to charge ratio of the unmodified peptide plus and minus the user defined tolerance in mass or mass to charge ratio units. Such a binary search will therefore return upper and lower bound peptides between which all the peptides listed in the peptide databank will match the mass or mass to charge ratio of the experimentally observed peptide (as unmodified according to the preferred embodiment) to within a certain tolerance.

By accurately measuring the mass to charge ratio of ions using, for example, a quadrupole Time of Flight mass spectrometer the number of possible peptides resulting from the two binary searches can, advantageously, be kept as small as possible because the user defined tolerance can be kept small. This initial list can then immediately be shortened into a short list since many of the peptides matching the mass or mass to charge ratio of the experimentally observed peptide (as unmodified according to the preferred embodiment) will not actually support the current combination of modifiers i.e. the peptides will not include the appropriate modifiable site(s).

The indexed peptide databank which is preferably searched against preferably consists of a linear array of records. Each record preferably consists of a number of fields which may include protein index information (i.e. information linking the peptide to a corresponding protein in a protein database) together with details of the accurate monoisotopic mass or mass to charge ratio of the peptide. Each peptide entry preferably also further includes information concerning the length and position of the peptide within the particular protein sequence.

The peptide records stored in the peptide databank are preferably sorted and arranged in order of mass or mass to charge ratio. Therefore, to find peptides which have a certain mass or mass to charge ratio to within a user defined tolerance, it is only necessary to do a binary search to find upper and lower bounds within the peptide databank.

If it is necessary to support multiple digests then an additional integer field may be included in each peptide record. Each bit in the additional integer field may be used as a flag to indicate that a given peptide is a product of a given digest. Additional fields may also include a digest probability to be incorporated into a protein scoring scheme.

The preferred searching method may be implemented using software code written, for example, using C/C++ programming language and compiled to run on a Microsoft Windows XP (RTM) platform using Microsoft Visual Studio (RTM) version 6.0. According to the preferred embodiment an indexed peptide databank based on a tryptic digest of a protein may be generated and loaded into the main memory of a computer. The peptide databank can then be searched against with possible peptides being initially filtered and short listed peptides subsequently characterised according to the preferred embodiment.

According to another embodiment a slightly different indexing scheme may be used wherein two index files are created. One of the index files contains just details of the accurate monoisotopic mass or mass to charge ratios of the peptides together with peptide index information which points to a corresponding second file. The second file may contain further information concerning the peptides i.e. protein index information and information concerning the length and position of the peptides within particular protein sequences.

The preferred embodiment treats individually tryptic peptides appearing multiple times (in the same or different protein entries) so the indexed peptide databank may contain many copies of the same peptide (especially for small peptides). Preferably, a shared memory model may be used so that only a single copy of the databank is maintained in the memory of the processing system.

According to an embodiment a mechanism for treating multiple digests may be implemented. The peptide record may, for example, contain a flag indicating the number of missed cleavages associated with the peptide.

With reference to FIG. 2 the process of peptide filtering as preferably implemented will now be described in more detail. Firstly, all combinations of user-selected modifiers are preferably enumerated. A standard algorithm for enumerating through all combinations may be used such as the algorithm described by Tucker in Applied Combinatorics (John Wiley & Sons Inc. 2001). This is a process of obtaining all combinations (of different sizes) with repeats. For a given number of modifiers n and a fixed number of modifiable sites k, the total number of combinations (i.e. of size k) is given by:
Cₖ=(n+k−1)!/(n−1)!k!

As the number of sites is unknown, it is necessary to consider the range of modifiable sites from zero up to some user specified maximum number of modifiable sites. So the total number of combinations to be considered per search against experimental data is given by:

$$C = {\sum }_{k = 0}^{k = {K}_{max}} {C}_{k}$$

For each of these combinations a modified mass or mass to charge ratio change is calculated and is preferably subtracted from the experimentally determined mass or mass to charge ratio of each peptide being analysed. The number of binary searches Bₛ of the indexed peptide databank which therefore need to be performed is:
Bₛ=2T_{q}C
where T_{q} is the total number of queries. A factor 2 is included since both an upper bound search and also a lower bound search are required. Searching of the databank preferably uses an iterative binary search to get the upper and lower bound peptides. These mass bounded peptides are then filtered.

The filtering stage preferably ensures that all possible peptides which then go on to be subsequently characterised at least contain the right number of residues and that the residues are of the right kind. The filtering stage therefore involves counting residues in the possible peptides and comparing the possible peptides with the current combination of modifiers. It is important that modifiers only apply to a single residue. Particular modifiers which may apply to several residues (degenerate modifiers) are refactored into several non-degenerate modifiers.

Accordingly, indexing peptides in the databank together with accurately measuring the mass to charge ratio of the peptides enables the first stage of processing to act as a high speed filter to substantially reduce the number of peptides which then need to be characterised in a second stage.

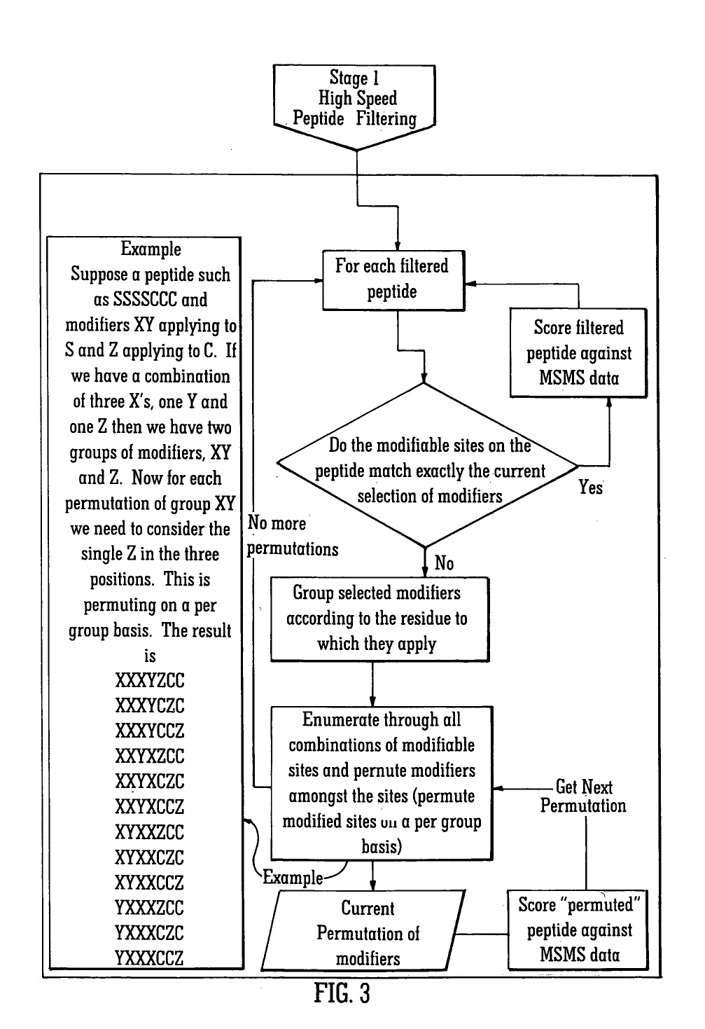

Once a short list of peptides is formed, a second characterisation stage is then performed on the short list of possible peptides. This characterisation stage is shown and described in more detail in relation to FIG. 3. The characterisation stage carries out possible permutations of the modifiers amongst modifiable sites on the peptide and scores each permutation against the experimentally observed fragment (MS/MS) mass spectral data.

The characterisation stage comprises a number of steps. Firstly, for each short listed or filtered peptide it is determined whether the modifiable sites on the peptide in question exactly match the current selection of modifiers. If they do, then the peptide is scored against the experimentally derived fragmentation or MS/MS data and then the next short listed peptide is considered.

If, however, the modifiable sites on the peptide do not exactly match the current selection of modifiers, then the selected combination of modifiers are preferably grouped according to the residue to which the modifiers apply. Then, all the combinations of modifiable sites are enumerated through. Modifiers amongst the sites are then preferably permuted with modified sites being permuted on a per group basis. This is because there may be more modifiable sites on the possible peptide than is required to modify. So given a number of sites which can be modified and the number of modifiers applied, all combinations of sites that can have the modifications applied are selected.

By way of illustration, the peptide PGPCCKDKCECAEGGCKT may be considered. It may be determined, for example, that C and K can be modified and a limitation of 4 modifiable sites may be imposed. The eight sites that can therefore be modified as they appear in order are CCKKCCCK. The various possibilities can therefore be enumerated as follows: (i) CCKK______ (represented in software by an array of size 4 as 0123); (ii) CCK_C______ (represented in software by an array of size 4 as 0124); (iii) CCK______C______ (represented in software by an array of size 4 as 0125); (iv) CCK______ C_(represented in software by an array of size 4 as 0126); (v) CCK______K (represented in software by an array of size 4 as 0127); (vi) CC_KC______ (represented in software by an array of size 4 as 0134) . . . and so on. However, it may be that only one K modification and three C modifications are allowed which means that some of the possibilities enumerated above such as (i) can be discarded.

For each valid modifiable site combination, the modifiers are then preferably permuted amongst the sites to which the modifiers apply (on a per group basis). So if M1 applies to C, M2 applies to C, M3 applies to C and M4 applies to K then M1, M2 and M3 must be applied in all the three positions and for each of these positions M4 must be applied to K. This is then repeated for the next enumeration. Algorithms to carry out such processes may use standard lexicographic algorithms such as those disclosed, for example, in Knuth “The Art of Computer Programming” Volume 4, (Pre-Fascicle 2b: Generating all permutations).

For each permutation of modifiers, the permuted peptide is scored against experimentally obtained fragmentation or MS/MS data and then the next permutation is considered. Once all permutations have been considered the next short listed peptide is considered.

Experimental results will now be presented which illustrate the improvement in overall searching time which is achievable according to the preferred embodiment. Two sets of results are presented. One set was obtained when only two modifiers were considered and the other set was obtained when four modifiers were considered.

Five randomly chosen proteins were considered. These five proteins comprised TCMO_MEDSA, SNC2_YEAST, DAK2_SCHPO, CSPB_BACGO and FMT_BOVIN. The proteins were theoretically digested using trypsin and one missed cleavage. Resultant peptides less than 8 residues in length or greater than 24 residues in length were discarded from further consideration leaving a total of 168 peptides to be considered. These remaining 168 peptides were then modified.

The sequences of the five randomly chosen proteins were read and then either two or four modifications were applied to appropriate residues in the sequence. In order to introduce a further random effect only those residues that had an even position within its peptide sequence were modified. The four possible modifications which were allowed for were Palmitoylation applying to threonine, Phosphoryl applying to serine, Carboxymethyl applying to cysteine, and Hydroxyl applying to aspartic acid.

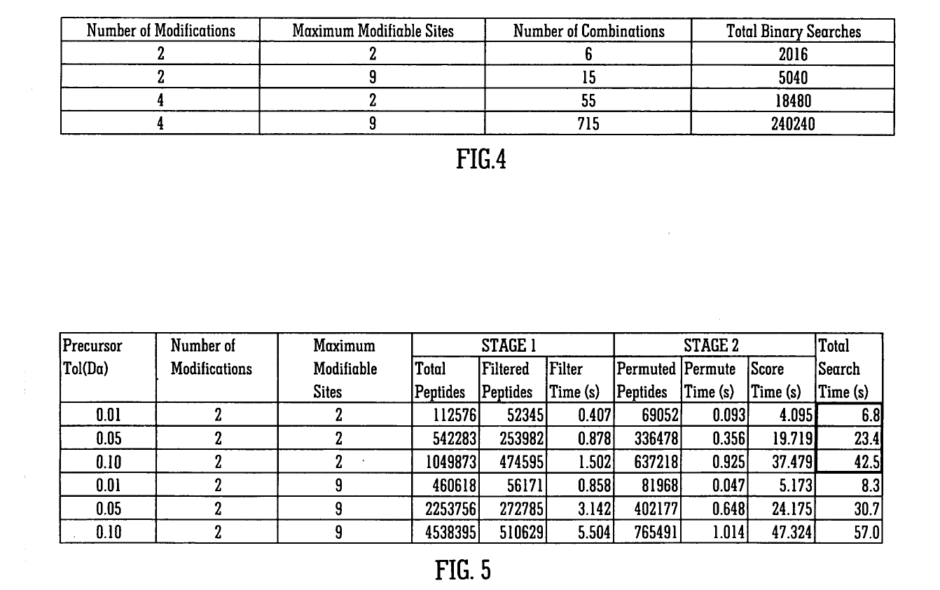

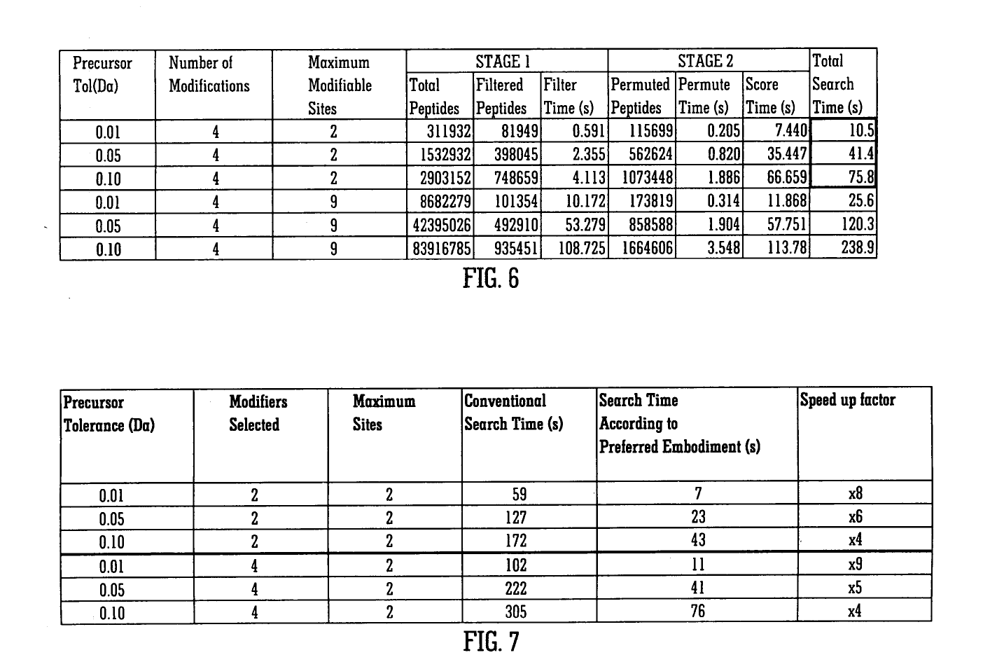

FIG. 4 shows the total number of combinations and binary searches required to search against the 168 peptides for different numbers of modifications and modifiable sites. FIG. 5 shows in more detail the results for two modifiers and FIG. 6 shows in more detail the results for four modifiers.

FIGS. 5 and 6 separately show the time taken for the first stage of initially filtering possible peptides and also the second stage of subsequently permuting and scoring peptides. The figures also show corresponding results when different mass tolerances and maximum number of modifiable sites were considered. The times were obtained by searching against a peptide databank derived from the Swiss Prot database containing 105224 entries and indexed using a typtic digest. Code was run on a Compaq Evo W6000 (RTM) running at 1.7 GHz in a single processor operation.

FIGS. 5 and 6 illustrate the time taken to filter peptides and the time taken to permute and score the final peptides i.e. all the potential combinations. It can be seen that as the number of modifiers and modifiable sites increases, significantly more peptides are initially returned which then need to be filtered. However, importantly, the number of peptides which pass through the filtering process do not increase at the same rate at which the total number of peptides increases at. As the total number of peptides to be considered increases, the filter time begins to become a more significant part of the overall search time. This is due to the fact that as the number of combinations to be considered increases, the same peptides will be assessed repeatedly in order to determine whether or not they can support the current combination. Similarly, the same peptides will repeatedly fail this test and will therefore not require scoring.

Widening the mass tolerance results in extra peptides being considered and passing through the filter. For example, when the mass tolerance is increased from 0.01 Da to 0.10 Da, then the total number of peptides to be considered increases by a factor of about ×10 and this results in a corresponding increase in the filter time. It can therefore be seen that it is particularly advantageous to accurately determine the mass or mass to charge ratio of the peptides to be analysed since this will reduce significantly the total number of peptides which need to be considered. Further improvements can be obtained by storing information concerning the number and type of residues a peptide has. For example, a memory cache may be used to avoid the repeated calculation and thus reduce the filter time and hence the total search time.

FIG. 7 shows comparative results for conventional search times against search times according to the preferred embodiment. It can be seen that the preferred embodiment enables the overall search time to be significantly sped up compared to conventional search times. The conventional search times were obtained using the Proteinlynx (RTM) Global Server 2.05B19. The search times according to the preferred embodiment are taken from some of the highlighted results presented in FIGS. 5 and 6. The search times listed in FIG. 7 which relate to the preferred embodiment include approximately 2 s to read the peptide data to be analysed.

From the results shown in FIG. 7, it can be seen that if the mass tolerance is increased, then there is a corresponding fall off in the speed up factor (or improvement) of the search time according to the preferred embodiment relative to the search time taken using conventional techniques. The reason for this fall off in improvement is not at present fully understood since presumably the conventional on the fly implementation must also have to deal with a correspondingly increased number of peptides at the wider mass tolerance. Nonetheless, the results show that the overall search time taken to analyse peptides according to the preferred embodiment can be significantly reduced compared to conventional search times especially when the mass or mass to charge ratio of the peptides is accurately determined i.e. the precursor or parent ion mass tolerance is kept small. The present invention therefore represents a significant improvement in the art.

The preferred embodiment is also capable of identifying proteins if the number of modifications of interest is set at zero.

Although the present invention has been described with reference to preferred embodiments, it will be understood by those skilled in the art that various changes in form and detail may be made without departing from the scope of the invention as set forth in the accompanying claims.

1. A method of identifying a modified protein, comprising:
digesting a modified protein to produce a plurality of peptides;
ionising at least one of said plurality of peptides to form one or more peptide ions;
mass analysing one or more of said peptide ions to determine the mass or mass to charge ratio of at least one of said peptides or peptide ions; and
identifying said modified protein by:
(i) determining the theoretical unmodified mass or mass to charge ratio which a peptide or peptide ion would have, had the protein from which said peptide or peptide ion is derived not been modified;
(ii) searching a databank of peptides using said theoretical unmodified mass or mass to charge ratio; and
(iii) determining peptides in said databank which have a mass or mass to charge ratio which corresponds with said theoretical unmodified mass or mass to charge ratio.

2. A method as claimed in claim 1, wherein said modified protein has been post-translationally modified.

3. A method as claimed in claim 1, wherein said modified protein has been modified by methylation, hydroxylation, oxidation, formylation, acetylation, carboxylation, phosphorlyation, sulphation, cysteinylation, glycosalation, farnesylation, myristoylation, biotinylation, palmitoylation or stearoylation.

4. A method as claimed in claim 1, further comprising determining, selecting or considering one or more modifications of interest.

5. A method as claimed in claim 4, wherein said modified protein is determined or considered to have been modified by at least one of said modifications of interest.

6. A method as claimed in claim 4, further comprising enumerating through at least one or more combinations of modifications of interest.

7. A method as claimed in claim 6, further comprising enumerating through all combinations of modifications of interest.

8. A method as claimed in claim 6, further comprising determining, selecting or considering a maximum number of modifications.

9. A method as claimed in claim 8, wherein the step of enumerating through combinations of modifications of interest comprises enumerating through combinations of modifications of interest up to said maximum number of modifications.

10. A method as claimed in claim 4, wherein said step of determining the theoretical unmodified mass or mass to charge ratio comprises calculating or determining the mass or mass to charge ratio of a combination of modifications of interest.

11. A method as claimed in claim 10, wherein said step of determining the theoretical unmodified mass or mass to charge ratio comprises altering, reversing, counteracting, negating, unmodifying or adjusting the mass or mass to charge ratio of said peptide or peptide ion in view of said combination of modifications of interest.

12. A method as claimed in claim 11, wherein said step of determining the theoretical unmodified mass or mass to charge ratio comprises subtracting the mass or mass to charge ratio of said combination of modifications of interest from the mass or mass to charge ratio of said peptide or peptide ion as determined by said step of mass analysing.

13. A method as claimed in claim 11, wherein said step of determining the theoretical unmodified mass or mass to charge ratio comprises adding the mass or mass to charge ratio of said combination of modifications of interest to the mass or mass to charge ratio of said peptide or peptide ion as determined by said step of mass analysing.

14. A method as claimed in claim 1, further comprising determining, selecting or considering an allowed mass or mass to charge ratio tolerance between the theoretical unmodified mass or mass to charge ratio of said peptide or peptide ion and the mass or mass to charge ratio of peptides listed in said databank.

15. A method as claimed in claim 14, wherein said mass tolerance falls within a range selected from the group consisting of: (i) <0.01 Da; (ii) 0.01-0.02 Da; (iii) 0.02-0.03 Da; (iv) 0.03-0.04 Da; (v) 0.04-0.05 Da; (vi) 0.05-0.06 Da; (vii) 0.06-0.07 Da; (viii) 0.07-0.08 Da; (ix) 0.08-0.09 Da; (x) 0.09-0.10 Da; and (xi) >0.10 Da.

16. A method as claimed in claim 14, wherein said mass to charge ratio tolerance falls within a range selected from the group consisting of: (i) ≦0.1 mass to charge ratio units; (ii) ≦0.01 mass to charge ratio units; (iii) ≦0.001 mass to charge ratio units; (iv) ≦0.0001 mass to charge ratio units; (v) ≦0.00001 mass to charge ratio units; and (vi) ≦0.000001 mass to charge ratio units.

17. A method as claimed in claim 14, wherein said step of searching a databank of peptides comprises determining or selecting peptides listed in said databank which have a mass or mass to charge ratio corresponding to the theoretical unmodified mass or mass to charge ratio to within said mass or mass to charge ratio tolerance.

18. A method as claimed in claim 17, further comprising determining a peptide in said databank of peptides which has a mass or mass to charge ratio corresponding to the theoretical unmodified mass or mass to charge ratio to within an upper limit of said mass or mass to charge ratio tolerance.

19. A method as claimed in claim 18, further comprising determining a peptide in said databank of peptides which has a mass or mass to charge ratio corresponding to the theoretical unmodified mass or mass to charge ratio to within a lower limit of said mass or mass to charge ratio tolerance.

20. A method as claimed in claim 19, further comprising selecting peptides in said databank of peptides having masses or mass to charge ratios between said upper limit and said lower limit.

21. A method as claimed in claim 20, wherein peptides in said databank of peptides having masses or mass to charge ratios between said upper limit and said lower limit form an initial list of peptides.

22. A method as claimed in claim 21, further comprising rejecting or discarding peptides from said initial list of peptides which do not support at least one of said combination of modifications of interest.

23. A method as claimed in claim 22, further comprising forming a shortlist of possible peptides.

24. A method as claimed in claim 23, wherein at least a majority of peptides in said shortlist of possible peptides have: (i) a mass or mass to charge ratio which corresponds to the theoretical unmodified mass or mass to charge ratio of said peptide or peptide ion to within said mass or mass to charge ratio tolerance; and (ii) at least the same number and type of modifiable sites in order to support at least one of said combination of modifications of interest.

25. A method as claimed in claim 23, further comprising determining whether the modifiable sites of a peptide in said shortlist of possible peptides exactly matches a combination of modifications of interest.

26. A method as claimed in claim 25, wherein if the modifiable sites of a peptide in said shortlist of possible peptides exactly or substantially matches a combination of modifications of interest then the peptide in said shortlist is scored against fragmentation or MS/MS mass spectral data corresponding to said peptide ion.

27. A method as claimed in claim 25, wherein if the modifiable sites of a peptide in said shortlist of possible peptides do not exactly or substantially match a combination of modifications of interest then the modifications of interest are grouped according to the residue to which they apply.

28. A method as claimed in claim 27, further comprising enumerating through combinations of modifiable sites.

29. A method as claimed in claim 28, further comprising permuting modifications of interest amongst the modifiable sites.

30. A method as claimed in claim 29, wherein said modifications of interest are permuted on a per group basis.

31. A method as claimed in claim 29, further comprising scoring each permuted peptide against fragmentation or MS/MS mass spectral data corresponding to said peptide ion.

32. A method as claimed in claim 1, wherein said databank comprises a databank of peptides derived from unmodified proteins.

33. A method as claimed in claim 32, wherein peptides listed in said databank are arranged in order of mass or mass to charge ratio.

34. A method as claimed in claim 33, wherein said peptides are arranged in order of monoisotopic mass or mass to charge ratio.

35. A method as claimed in claim 1, wherein said step of identifying said modified protein further comprises repeating steps (i), (ii) and (iii) for a plurality of different peptides or peptide ions.

36. A mass spectrometer comprising:
an ion source for ionising at least one of a plurality of peptides derived from the digestion of a modified protein to form one or more peptide ions;
a mass analyser for mass analysing one or more of said peptide ions to determine the mass or mass to charge ratio of at least one of said peptides or peptide ions; and
identifying means for identifying said modified protein, wherein, in use, said identifying means:
(i) determines the theoretical unmodified mass or mass to charge ratio which a peptide or peptide ion would have, had the protein from which said peptide or peptide ion is derived not been modified;
(ii) searches a databank of peptides using said theoretical unmodified mass or mass to charge ratio; and
(iii) determines peptides in said databank which have a mass or mass to charge ratio which corresponds with said theoretical unmodified mass or mass to charge ratio.

37. A method of identifying a modified protein, comprising:
(i) determining the theoretical unmodified mass or mass to charge ratio which a peptide or peptide ion would have, had the protein from which said peptide or peptide ion is derived not been modified;
(ii) searching a databank of peptides using said theoretical unmodified mass or mass to charge ratio; and
(iii) determining peptides in said databank which have a mass or mass to charge ratio which corresponds with said theoretical unmodified mass or mass to charge ratio.

38. Apparatus for identifying a modified protein, wherein, in use, said apparatus:
(i) determines the theoretical unmodified mass or mass to charge ratio which a peptide or peptide ion would have, had the protein from which said peptide or peptide ion is derived not been modified;
(ii) searches a databank of peptides using said theoretical unmodified mass or mass to charge ratio; and
(iii) determines peptides in said databank which have a mass or mass to charge ratio which corresponds with said theoretical unmodified mass or mass to charge ratio.

39. A method of identifying a modified protein, comprising:
(a) determining, selecting or considering one or more modifications of interest;
(b) enumerating through one or more combinations of modifications of interest;
(c) calculating the mass or mass to charge ratio of one or more of said combinations of modifications of interest;
(d) subtracting the calculated mass or mass to charge ratio of one of said combinations of modifications of interest from the experimentally determined mass or mass to charge ratio of a peptide or peptide ion to determine a theoretical unmodified mass or mass to charge ratio which a peptide or peptide ion would have, had the protein from which said peptide or peptide ion is derived not been modified;
(e) searching a databank of peptides using said theoretical unmodified mass or mass to charge ratio to return an initial list of peptides;
(f) discarding from said initial list of peptides, peptides which cannot support the combination of modifications of interest thereby producing a short list of peptides; and
(g) scoring peptides in said short list of peptides against fragmentation mass spectral data.

40. Apparatus for identifying a modified protein, wherein, in use, said apparatus:
(a) determines, selects or considers one or more modifications of interest;
(b) enumerates through one or more combinations of modifications of interest;
(c) calculates the mass or mass to charge ratio of one or more of said combinations of modifications of interest;
(d) subtracts the calculated mass or mass to charge ratio of one of said combinations of modifications of interest from the experimentally determined mass or mass to charge ratio of a peptide or peptide ion to determine a theoretical unmodified mass or mass to charge ratio which a peptide or peptide ion would have, had the protein from which said peptide or peptide ion is derived not been modified;
(e) searches a databank of peptides using said theoretical unmodified mass or mass to charge ratio to return an initial list of peptides;
(f) discards from said initial list of peptides, peptides which cannot support the combination of modifications of interest thereby producing a short list of peptides; and
(g) scores peptides in said short list of peptides against fragmentation mass spectral data.
